(19) United States
(12) Patent Application Publication
(10) Pub. No.: US 2024/0096494 A1
(43) Pub. Date: Mar. 21, 2024

(54) WEARABLE DEVICE, METHOD OF OPERATING WEARABLE DEVICE, AND NON-TRANSITORY COMPUTER READABLE MEDIUM

(71) Applicant: FUJIFILM Corporation, Tokyo (JP)

(72) Inventor: Noriaki IDA, Tokyo (JP)

(73) Assignee: FUJIFILM Corporation, Tokyo (JP)

(21) Appl. No.: 18/469,513

(22) Filed: Sep. 18, 2023

(30) Foreign Application Priority Data
Sep. 21, 2022 (JP) 2022-150278

(51) Int. Cl. G16H 50/20; A61B 5/00; A61B 5/117; G06F 21/32; G16H 40/67; G16H 80/00

(52) U.S. Cl. CPC G16H 50/20; A61B 5/117; A61B 5/681; G06F 21/32; G16H 40/67; G16H 80/00

(57) ABSTRACT

A smart watch which is a wearable device collects data while worn including at least biological information of a wearer in a healthy state, stores approval information for controlling output of the data while worn by prior approval of the wearer, receives a data provision request for a medical device that generates medical diagnostic information, and provides the data while worn to the medical device based on the approval information and the data provision request. A doctor makes a medical diagnosis using the data while worn acquired by the medical device and examination data.

### CROSS-REFERENCE TO RELATED APPLICATIONS

This application claims priority under 35 U.S.C § 119(a) to Japanese Patent Application No. 2022-150278 filed on 21 Sep. 2022. The above application is hereby expressly incorporated by reference, in its entirety, into the present application.

### BACKGROUND OF THE INVENTION

### 1. Field of the Invention

The present invention relates to a wearable device, a method of operating a wearable device, and a non-transitory computer readable medium.

### 2. Description of the Related Art

A wearable device such as a smart watch can measure and record biological information of a device wearer. In a case of receiving a medical diagnosis at a hospital or the like, a doctor may combine biological information in a case of diagnosing a disease or injury with biological information collected in a healthy state to enable a more accurate diagnosis. In addition, it becomes easy for the hospital to acquire information such as an exact time at which an abnormality such as the disease or injury is considered to have occurred, based on biological information continuously measured by the wearable device.

For example, in JP2022-037048A (corresponding to US2019/103007A1), a change in an orientation of a mobile device, an altitude of the device, an acceleration, and a heart rate are measured to detect that a user has fallen. Further, it is described that, in a case where a fall is detected, a notification is given to another person such as a doctor. JP2022-025095A (corresponding to US2017/372497A1) describes a trained model that determines the presence or absence of an abnormality from a medical image of a subject by using machine learning, in which a body mass index, a blood pressure, a heart rate, a respiratory rate, and the like of a subject are also input as non-image inputs to determine the presence or absence of the abnormality. JP2022-003554A (corresponding to US2018/329584A1) describes that collection/sharing of personal information data such as information on a level of health or fitness of a user is performed after notifying a user and obtaining consent.

### SUMMARY OF THE INVENTION

However, since biological information recorded by a wearable device includes those that are applicable to personal information, it is necessary to obtain approval from a device wearer himself/herself for viewing data of another person or providing data to another person. On the other hand, it is cumbersome and time-consuming to approve viewing by a medical worker and provision of data to a medical worker each time it is necessary. In addition, there is a problem in that in such a case where a device wearer loses consciousness while going out and falls down and is transported by ambulance to a hospital, it is not possible to approve a permission request to view or provide data.

An object of the present invention is to provide a wearable device, a method of operating a wearable device, and a non-transitory computer readable medium capable of safely and promptly providing data collected in a healthy state even in a state in which a wearer is unconscious.

An aspect of the present invention is a wearable device comprising a processor, in which the processor collects data while worn including at least biological information of a wearer in a healthy state, stores approval information for controlling output of the data while worn by prior approval of the wearer, receives a data provision request for a medical device that generates medical diagnostic information, and outputs the data while worn to the medical device based on the approval information and the data provision request.

A wearable device according to a second aspect classifies the data while worn for each measurement item, and stores the approval information in which whether or not to provide data is set for each measurement item, in the wearable device according to the first aspect.

A wearable device according to a third aspect stores the approval information in which an expiration date is set, gives a notification at an end of the expiration date, and receives an update operation of the expiration date, in the wearable device according to the first or second aspect.

A wearable device according to a fourth aspect acquires medical device information from the medical device, and provides the data while worn to the medical device based on the approval information and the medical device information, in the wearable device according to any one of the first to third aspects.

A wearable device according to a fifth aspect acquires affiliation information of the medical device from the medical device information, and provides the data while worn according to the affiliation information, in the wearable device according to the fourth aspect.

A wearable device according to a sixth aspect discriminates the medical device for performing machine learning from the medical device information, and provides the data while worn for generation of the medical diagnostic information using the machine learning, in the wearable device according to the fourth or fifth aspect.

A wearable device according to a seventh aspect discriminates the medical device for performing machine learning from the medical device information, and provides the data while worn for a re-training process in the machine learning, in the wearable device according to any one of the fourth to sixth aspects.

A wearable device according to an eighth aspect performs biometric authentication of the wearer, and receives the prior approval in a case where the wearer is determined by the biometric authentication, in the wearable device according to any one of the first to seventh aspects.

A wearable device according to a ninth aspect displays the approval information on a screen by the biometric authentication, in the wearable device according to the eighth aspect.

A wearable device according to a tenth aspect creates the approval information using proxy approval by a proxy different from the wearer, instead of the prior approval, in the wearable device according to any one of the first to ninth aspects.

A wearable device according to an eleventh aspect registers in advance an external input device capable of creating the approval information, receives the approval information from the external input device, and provides the data while worn, in the wearable device according to any one of the first to tenth aspects.

A wearable device according to a twelfth aspect stores the data while worn in an external storage device, and provides the data while worn to the medical device from the external storage device in response to the approval information and the data provision request, in the wearable device according to any one of the first to eleventh aspects.

A wearable device according to a thirteenth aspect permits access to the external storage device and displays access means on a screen by biometric authentication or a specific operation by a medical worker based on the approval information, in the wearable device according to the twelfth aspect.

A method of operating a wearable device according to a fourteenth aspect comprises a step of collecting data while worn including at least biological information of a wearer in a healthy state, a step of storing approval information for controlling output of the data while worn by prior approval of the wearer, a step of receiving a data provision request for a medical device that generates medical diagnostic information, and a step of providing the data while worn to the medical device based on the approval information and the data provision request.

A non-transitory computer readable medium for storing a computer-executable program according to a fifteenth aspect causes a computer provided in a wearable device to execute a function of collecting data while worn including at least biological information of a wearer in a healthy state, a function of storing approval information for controlling output of the data while worn by prior approval of the wearer, a function of receiving a data provision request for a medical device that generates medical diagnostic information, and a function of providing the data while worn to the medical device based on the approval information and the data provision request.

According to the present invention, it is possible to safely and promptly provide data collected in a healthy state even in a state in which a wearer is unconscious.

### BRIEF DESCRIPTION OF THE DRAWINGS

FIG. 1 is an external view of a smart watch.

FIG. 2 is an external view of a back surface of a smart watch main body.

FIG. 3 is a block diagram showing functions of the smart watch main body.

FIG. 4 is an explanatory diagram showing a relationship of performing a data provision request and provision of data while worn between the smart watch and a medical device.

FIG. 5 is an explanatory diagram for providing the medical device with the data while worn measured by the smart watch and medical diagnostic information generated in a medical facility.

FIG. 6 is an explanatory diagram of a biometric authentication screen that receives authentication of biological information.

FIG. 7 is an explanatory diagram of an initial state of an approval information registration screen for performing prior approval.

FIG. 8 is an explanatory diagram of the approval information registration screen in a case of performing usage setting in prior approval.

FIG. 9 is an explanatory diagram of the approval information registration screen in a case of performing provision destination setting of the data while worn in prior approval.

FIG. 10 is an explanatory diagram of the approval information registration screen in a case of performing individual approval of contents of the data while worn to be provided in prior approval.

FIG. 11 is an explanatory diagram showing a detailed setting of an expiration date of approval information, which is set on the approval information registration screen.

FIG. 12 is an explanatory diagram showing a screen for notifying of an end of an expiration date of approval information and prompting update.

FIG. 13 is an explanatory diagram showing a state in which the smart watch and the medical device are connected in an emergency medical mode.

FIG. 14 is a flowchart showing a series of flows of the data provision request and the provision of the data while worn.

FIG. 15 is an explanatory diagram in which (A) represents registration of external device information in a setting mode, (B) represents a movement of a data provision request in an emergency medical mode, and (C) represents a movement of data provision, in a second embodiment.

FIG. 16 is an explanatory diagram in which (A) represents registration of the external device information and transmission of the approval information in a setting mode, (B) represents a movement of storage of the data while worn in a normal mode, and (C) represents a movement of a data provision request and data provision in an emergency medical mode, in a third embodiment.

FIG. 17 is an explanatory diagram showing an access permission screen for access to an external storage device in the third embodiment.

### DESCRIPTION OF THE PREFERRED EMBODIMENTS

### First Embodiment

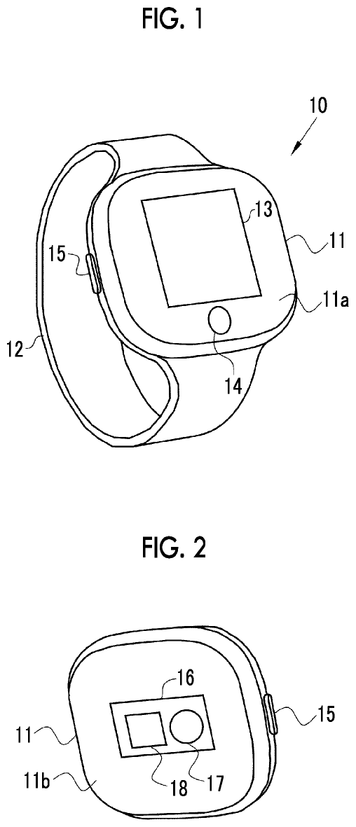

As shown in FIG. 1, a smart watch 10 is a wristwatch-type wearable device and is composed of a smart watch main body 11 and a belt 12. The smart watch main body 11 is physically connected to the belt 12 and is fixed to an arm of a wearer who is a user with an appropriate strength by adjusting a length of the belt 12. At least a display 13 and a camera 14 are provided on a main body front surface 11a which is an outer side during wearing and is visible to the wearer in the smart watch main body 11. The display 13 is preferably a touch screen having a position input function. The camera 14 only needs to be provided on the main body front surface 11a and may be located at an upper end or a right end other than a lower end as shown in FIG. 1. It is preferable that an operation button 15 is provided at any position on the main body front surface 11a or a main body side surface. The operation button 15 can receive a power on/off operation and receive an operation of switching between a normal mode and an emergency medical mode. A plurality of the operation buttons 15 may be provided. Further, although not shown, a speaker and a microphone are also provided.

As shown in FIG. 2, a biological information sensor 16 that measures biological information of the wearer is provided on a main body back surface 11b of the smart watch main body 11, which comes into contact with the arm of the wearer. In FIG. 2, the belt 12 is not shown. The biological information sensor 16 includes at least one light emitting portion 17 and at least one light receiving portion 18. The light emitting portion 17 emits special light such as green LED light emission, and the light receiving portion 18 receives return light in a case where an arm portion of the wearer is irradiated with the special light. The biological information sensor 16 analyzes the received light to acquire the biological information such as a heartbeat, a blood pressure, and an oxygen saturation of the wearer in real time.

In addition to the biological information sensor 16, the smart watch 10 also comprises an acceleration sensor, a vibration sensor, a temperature sensor, and the like, and may estimate an amount of exercise by combining a plurality of types of data including movement information and the like. Various functions of the smart watch 10 described below will be realized by the smart watch main body 11 in a state in which the smart watch 10 is normally worn on the arm of the wearer by the belt 12.

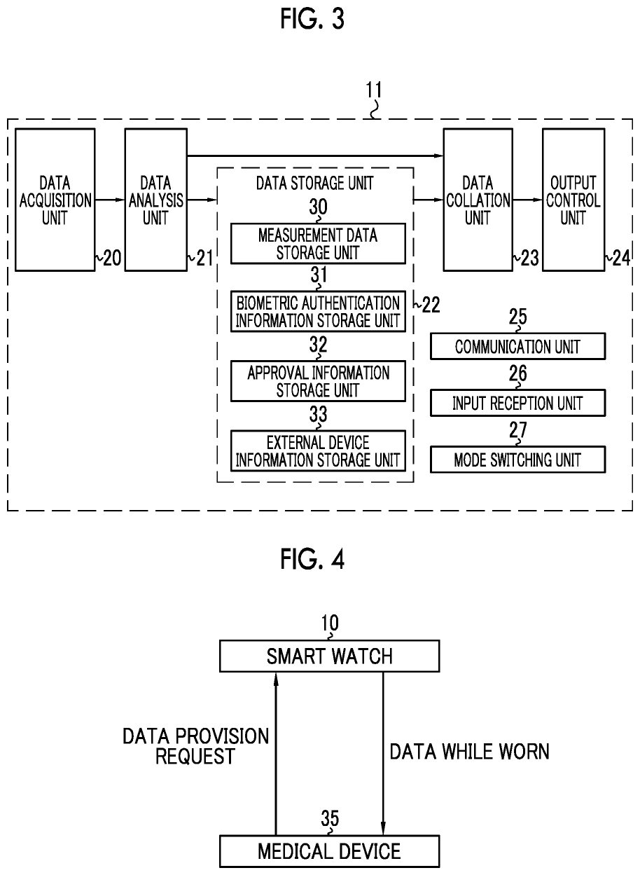

As shown in FIG. 3, the smart watch main body 11 according to the embodiment of the present invention is a computer such as a personal computer or a workstation in which an application program for realizing a predetermined function is installed. The computer comprises a central processing unit (CPU) which is a processor, a memory, a storage, and the like and realizes various functions of a data acquisition unit 20, a data analysis unit 21, a data storage unit 22, a data collation unit 23, an output control unit 24, a communication unit 25, an input reception unit 26, and a mode switching unit 27 by a program stored in the storage. The wearable device uses the above functions to provide data in response to a data provision request even in a state in which the wearer is unconscious.

The data acquisition unit 20 acquires data while worn including biological information in a healthy state of the wearer who wears the smart watch 10 by measurement of various sensors such as the biological information sensor 16 and transmits the data while worn to the data analysis unit 21. The biological information includes a pulse, a heart rate, a blood pressure, a blood oxygen concentration, and the like. In addition, a body temperature may be acquired by the temperature sensor, and movement information of the wearer may be acquired from the vibration sensor, the acceleration sensor, or the like.

The data analysis unit 21 analyzes and extracts various types of data acquired by the data acquisition unit 20 and digitizes the data. The data analysis unit 21 also has an image processing function of performing conversion and analysis of image information captured by the camera 14. Image processing includes creation of a still image or a moving image, as well as extraction of face information in face authentication and extraction of iris information in iris authentication. The extraction is to detect features specific to an imaging target and specify information that can be used for biometric authentication which will be described later. Voice information in voice authentication is also a feature of an individual and is extracted as information that can be used for authentication. Further, a plurality of types of data may be combined to acquire information such as calorie consumption as data included in the data while worn. The data obtained by the analysis is transmitted to the data storage unit 22 or the data collation unit 23.

The data storage unit 22 stores data received from the data analysis unit 21 or data input via the communication unit 25 or the input reception unit 26 in a storage memory (not shown) or the like. The data is distributed to a measurement data storage unit 30 that stores biological information in association with a date and time, a biometric authentication information storage unit 31 that stores biometric authentication information for performing setting of approval information and viewing or outputting of personal information, an approval information storage unit 32 that stores approval information for providing data in an emergency, which is input by a wearer in advance, and an external device information storage unit 33 that stores medical devices of a medical facility and terminals of a wearer, his/her family, and the like, which are registered in advance.

In a case where a data provision request is received from a medical facility, the data collation unit 23 collates request contents with the data while worn stored in the measurement data storage unit 30 and the approval information stored in the approval information storage unit 32, and selects the data to be output. The selected data is transmitted to the output control unit 24. It is preferable that the data collation unit 23 includes a temporary storage memory and temporarily stores input information.

The output control unit 24 performs control of contents to be displayed on the display 13, control of contents that the communication unit 25 transmits the data to the external device, and control of contents of a voice output by the speaker. It is preferable that the display 13 displays that the data while worn to be provided in response to the data provision request is provided by wireless or wired communication and that the data provision is performed. The past data or real-time data of the biological information acquired in response to an instruction of the wearer can be output to the display 13 and checked at will.

The communication unit 25 is an interface that performs communication processing with an external device by wire or wirelessly and exchanges information with the external device. The communication unit 25 receives the data provision request for the data while worn by the medical facility or the like. In addition, the communication unit 25 transmits the data while worn to be provided from the output control unit 24 to the medical facility or the like in response to the data provision request.

The input reception unit 26 is an input interface that receives various operation inputs. The input reception unit 26 receives and recognizes inputs from a touch screen provided on the main body front surface 11a as the display 13, the operation button 15, and the microphone (not shown) which are provided on the smart watch main body 11. The display 13 may not have a function of the touch screen, but in such a case, a plurality of the operation buttons 15 for performing user input are provided.

The mode switching unit 27 switches between the setting mode, the normal mode, and the emergency medical mode to realize functions of the smart watch 10. The setting mode is switched in a case of initial setting or by a user operation and sets the approval information. The normal mode is a basic state after the initial setting, and collects the data while worn. The emergency medical mode is switched by detecting an abnormality of the wearer or a specific operation and provides the data while worn.

The measurement data storage unit 30 stores the information acquired by various sensors and analyzed as the data while worn in association with date/time information which is a basic function of the smart watch. The data while worn includes data in a healthy state and data in a state in which a physical condition has changed due to an injury or illness, which are acquired in a state in which the wearer wears the smart watch 10. In addition to the information acquired from various sensors, physical feature information such as date of birth, height, and weight of the wearer, as well as medical records such as medication history, medical history, hospital visit history, primary care hospital, and attending physician can be stored. In addition, information calculated from a plurality of pieces of measurement information, such as calorie consumption and an amount of exercise, may be stored.

The biometric authentication information storage unit 31 stores biometric authentication information such as face authentication information, fingerprint authentication information, voice authentication information, and iris authentication information. The wearer registers the biometric authentication information in a case of purchasing the smart watch 10 and stores the biometric authentication information in the biometric authentication information storage unit 31. The smart watch 10 performs biometric authentication of a wearer for checking whether or not a person performing an approval operation is the wearer himself/herself in a case of performing prior approval of data provision of data while worn including biological information which is personal information.

The approval information storage unit 32 stores the approval information for data provision input in advance by the wearer. The approval information includes a range of data to be provided, usage, and information on a medical facility or medical device to which the information is provided. For example, it is possible to arbitrarily set and store the approval information such as approval of data provision of only blood pressure or approval of data provision to a designated medical facility, among pieces of the data while worn. In addition, since the physical feature information of the wearer and the medical record may correspond to personal information like the biological information, it is preferable to approve the data provision in the same manner as the biological information.

The external device information storage unit 33 stores information on external devices such as a medical device having a communication function recognized through the communication unit 25, an information communication terminal such as a personal computer (PC) or a smartphone for which the wearer has given permission, and a storage device. Thus, data communication can be automatically performed by connecting to the stored external device. In addition, in a case of being electrically connected to the medical device, the medical device information may be acquired from the medical device. The medical device information includes affiliation information of a medical facility to which the medical device belongs, a doctor who manages the medical device, usage of the data while worn, a type of information to be handled, and the like.

As shown in FIG. 4, in a case where a data provision request is received from a medical device 35 in the medical facility, the smart watch 10 can transmit the data while worn to the medical device 35. Specifically, the smart watch 10 that collects and stores the data while worn including at least biological information in a healthy state of the wearer receives the data provision request from the medical device 35 which is electrically connected. The smart watch 10 provides the data while worn that can be provided in response to a data provision request. The data while worn that can be provided is data for which the wearer has previously approved the provision of data based on the approval information stored in the approval information storage unit 32. The medical device 35 to which the smart watch 10 is connected is a device that generates medical diagnostic information or a PC that is provided in a medical facility for communicating the medical diagnostic information.

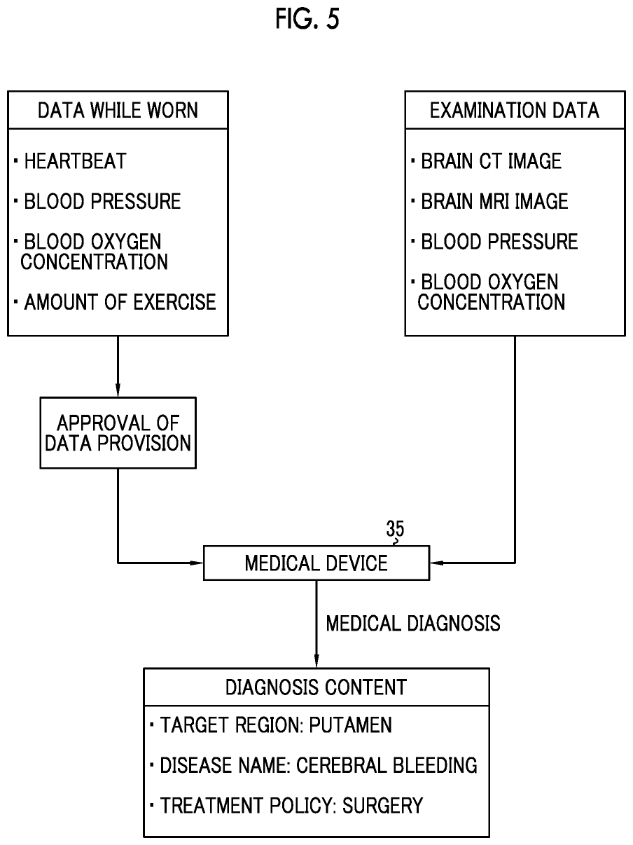

As shown in FIG. 5, the data while worn provided by the function of the smart watch 10 can be used to generate medical diagnostic information in a medical facility. In a case of providing the data, it is necessary to approve the data provision of the data while worn which is personal information. The provided data while worn is combined with examination data of the wearer (for example, a CT image or MRI image of a head) measured and acquired by various medical examinations at a medical facility and is used to specify a region to be treated, specify a disease name, and determine a treatment policy. For example, a doctor diagnoses a target region as “putamen”, a disease name as “cerebral bleeding”, and a treatment policy as “surgery” using the data while worn and the examination data which are medical diagnostic information. Since a change in blood pressure occurs in cerebral bleeding, the doctor can specify a bleeding time by obtaining blood pressure data in a case where the blood pressure changes from a healthy state in the data while worn, and diagnosis contents become more accurate. The medical diagnostic information may be generated using a device that uses machine learning.

Next, an operation of the smart watch 10 in the first embodiment will be described. The wearable device such as the smart watch 10 is a device that can collect and store data in a healthy state of the wearer, and comprises a setting mode for setting and storing approval information for controlling output of the data while worn by prior approval of the wearer, a normal mode for collecting the data while worn including at least biological information of the wearer in a healthy state, and an emergency medical mode for providing the data while worn to the medical device 35 that generates medical diagnostic information such as a medical facility based on the stored approval information and the recognized data provision request.

In the setting mode, it is preferable that the wearer performs registration of wearer information, determination of a biometric authentication method, registration of the biometric authentication information, and setting of the approval information in a case of purchasing the smart watch 10. The wearer can update or change contents registered in the setting mode at any timing.

The wearer information is information that is preferably associated with the data while worn in a case of providing data such as date of birth, height, weight, blood type, emergency contact information, medical history, and allergy information of the wearer, and is Information about a health of the wearer or information that may be needed in an emergency.

The biometric authentication information is information used for biometric authentication in a case of unlocking a lock screen of the display 13 or permitting viewing or output of personal information, and is used to check whether or not a person operating the smart watch 10 is a registered person. In the biometric authentication, the features of the individual analyzed by the data analysis unit 21 are collated with the biometric authentication information stored in the biometric authentication information storage unit 31, and it is determined whether or not the individual is the person in question. Specifically, in a case in which a collation result is a match or a match rate equal to or greater than a threshold value, it is determined that the operator is a person having the biometric authentication information stored in the biometric authentication information storage unit 31.

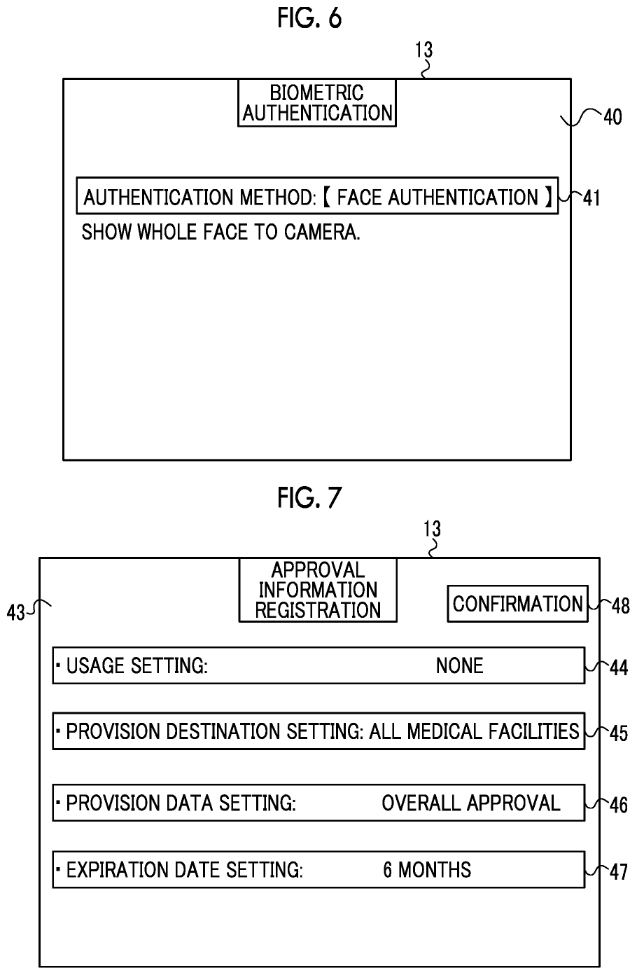

As shown in FIG. 6, in a case of registering the biometric authentication information, a biometric authentication screen 40 is displayed on the display 13. On the biometric authentication screen 40, an authentication method switching field 41 for selecting an authentication method and a message prompting the wearer to input biometric authentication information according to the authentication method are displayed. For example, in a case where the authentication method is face authentication, “face authentication” is displayed in the authentication method switching field 41, and a message prompting authentication using the camera 14 is displayed. In addition, in a case of being switched to the biometric authentication screen 40, the camera 14 is activated to image a face of the wearer, and a feature portion of the face is extracted by the data analysis unit 21 and stored in the biometric authentication information storage unit 31. A plurality of types of biometric authentication information can also be registered. In addition, the biometric authentication information of a proxy such as a guardian may be registered in addition to the wearer himself/herself.

In a case where the biometric authentication is performed using the biological information registered in the biometric authentication information storage unit 31, as in FIG. 6, the biometric authentication screen 40 is displayed on the display 13 and the input of the biological information is received. The wearer performs biometric authentication using the displayed authentication method. In a case where the wearer is determined to be the person in question as a result of the biometric authentication, the display indicates that the wearer is determined to be the person in question and switches to a next screen, for example, an approval information registration screen 43 for receiving the prior approval. In a case where the wearer is not determined to be the person in question, the input is received again.

In the biometric authentication, in a case where the result of not being determined to be the person in question reaches a certain number of times such as 5 times or 10 times, a biometric authentication function may be locked for a certain period such as 30 minutes or 1 hour. In addition, in a case where a plurality of biometric authentication information are registered, the biometric authentication may be performed using another authentication method by operating the authentication method switching field 41. Other authentication methods for face authentication include fingerprint authentication, iris authentication, voice authentication, and the like.

In a case where the authentication method is fingerprint authentication, the smart watch 10 comprises a fingerprint sensor (not shown) on the main body front surface 11a or a main body side surface, and the wearer contacts the fingerprint sensor with a finger registered in advance to input biological information of the fingerprint. The finger to be registered is preferably a thumb or index finger of a hand not wearing the smart watch 10. In addition, a plurality of fingers may be used.

In a case where the authentication method is iris authentication, the smart watch 10 adjusts a distance between the camera 14 and a face of the wearer in the same manner as in the face authentication. In order to image the iris, the wearer moves close enough to look into the camera 14 and to cause the iris to be imaged. The camera 14 images the face of the wearer, the data analysis unit 21 extracts a feature portion of the iris, and biological information of the iris is input.

In a case where the authentication method is voice authentication, the smart watch 10 comprises a microphone (not shown), and the wearer speaks a specific phrase registered in advance toward the microphone to input a voice and performs the voice authentication. The specific phrase to be registered is preferably a word or a sentence capable of identifying a person. Specific phrases include, for example, “approval”, “open sesame”, and “today is a sunny day”. A feature portion of the voice is extracted from the input voice by the data analysis unit 21, and biological information of the voice is input.

The approval information is information that is stored in the worn smart watch 10 and sets a condition for data to be provided in response to a request for provision of the data while worn in a case where the wearer is transported to a medical facility such as a hospital in a state in which the wearer has lost consciousness. The prior approval is performed by setting the approval information in advance in the setting mode by the wearer. The approval information may be approved for data provision without any particular limitation, or contents of the data provision to be approved may be individually designated. Specifically, the approval information includes the measurement items of the data while worn, the medical facility to which the data is provided, the usage of the data to be provided, and the like. In addition, a proxy such as a guardian may set the approval information instead of the wearer himself/herself.

In the setting mode, it is preferable that the registration of the approval information requires the wearer to be determined as the person in question by the biometric authentication before such registration. In the initial setting in a case of purchasing the smart watch, the biometric authentication information of the wearer is registered first, and the approval information is registered in a state in which the biometric authentication can be performed. In a case of registering the approval information, the content to be approved is set from the approval information registration screen 43. The biometric authentication is performed at least in a case of opening the approval information registration screen 43. In addition, the biometric authentication may be performed also in a case of confirming the input contents of the approval information. In a case where the approval information is changed after the initial setting, for example, a menu screen of the normal mode is switched to the setting mode by a predetermined operation, and the approval information registration screen 43 is displayed.

As shown in FIG. 7, in the registration of the approval information, the approval information registration screen 43 on the display 13 receives a setting of usage of the data while worn in a case of providing the data, a setting of a data provision destination, and a setting for each measurement item of the data to be provided, and a setting of an expiration date of the registered approval information and displays the setting contents. The approval information registration screen 43 includes a usage setting field 44, a provision destination setting field 45, a provision data setting field 46, and an expiration date setting field 47, and setting contents are displayed in each setting field.

The display of each setting field on the approval information registration screen 43 shown in FIG. 7 is setting contents in an initial state in a case of purchasing the smart watch, and the wearer selects each setting field and makes detailed settings. In the initial state, the usage designation of the data to be provided is set to “None” in the usage setting field 44, the setting of the provision destination such as the medical facility to which the data is provided is set to “all medical facilities” in the provision destination setting field 45, the measurement item of the data while worn to be provided is set to “overall approval” in the provision data setting field 46, and the expiration date of the approval information is set to “6 months” in the expiration date setting field 47. In a case where the setting is not changed, a confirmation button 48 is selected and stored in the approval information storage unit 32 as prior approval information.

The usage and the provision destination of the data while worn and the measurement items of the data while worn, which are set as the approval information, are collated with the medical device information received together with the data provision request in the emergency medical mode described later. Based on the collation result, whether or not to provide the data is discriminated, and the data while worn to be provided is selected from the contents of the data provision request.

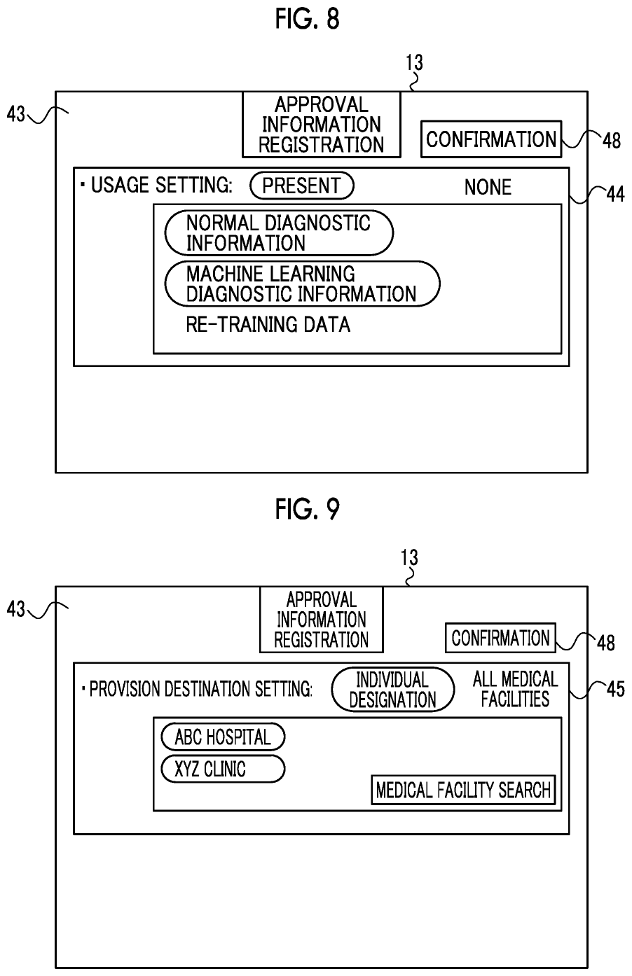

As shown in FIG. 8, it is possible to set the usage of the data while worn from the usage setting field 44. The wearer can change the display contents of the usage setting field 44 such that detailed settings can be made by selecting “Present” for the usage setting, and can set whether or not to individually approve items of the usage. The usage includes, for example, “normal diagnostic information generation” which is at least generation of medical diagnostic information that is not machine learning, “machine learning diagnostic information generation” which is generation of medical diagnostic information using machine learning, and “re-training data” which is different from the medical diagnostic information and provides the data while worn for a re-training process of machine learning in the medical device. The machine learning will be described later. Among these, in a case where the data provision for the generation of the normal diagnostic information and the machine learning diagnostic information is approved and the data provision is not approved for the re-training process of the machine learning, items of the “normal diagnostic information generation” and the “machine learning diagnostic information generation” are selected. The selected items are highlighted so as to be clearly distinguished from the non-selected item. The setting of the usage is not limited to the above contents, and other usages may be set. The “re-training data” may further enable setting of the data provision according to items of training contents of the re-training process.

The medical device 35 using machine learning has a computer algorithm consisting of a neural network, and performs inferences such as detection of lesions and classification of lesion types in input data according to training contents. The machine learning requires a large amount of data, and it is expected to improve the training accuracy by automatically acquiring data from a device installed in a medical facility such as a hospital and adding the data to training. In the re-training process, the collected (provided) data is used for training for improving the accuracy of the medical device 35 using machine learning, and the data used for the training and the training result may be fed back to a manufacturer of the medical device 35. Therefore, the setting of the “re-training data” for the usage includes providing the data while worn not only to all medical facilities but also to the manufacturer of the medical device 35.

In addition, in a case where the “re-training data” is selected for the setting of the usage shown in FIG. 8, a specific medical device manufacturer that sells the medical device 35 using machine learning may be designated to set whether or not to provide data, separately from the training contents. For example, a plurality of medical device manufacturers are listed and registered in the smart watch 10 in advance from the time of sale, and the wearer sets the medical device manufacturer by selecting from the list.

As shown in FIG. 9, in the provision destination setting field 45, the data provision destination can be individually set instead of “all medical facilities” for the provision destination setting. The setting of the provision destination of the data while worn can be designated by a medical facility name such as “ABC hospital” and “XYZ clinic”. The medical facility name of the provision destination may be listed and registered in the smart watch 10 in advance from the time of sale, and may be determined by the wearer by selecting from the list. It is preferable that the list is periodically updated to the latest list by downloading via the communication unit 25 or the like. In addition, the provision destination may be set in conjunction with a map application or the like provided in the smart watch 10, or a medical facility search function that allows a search from text input may be provided.

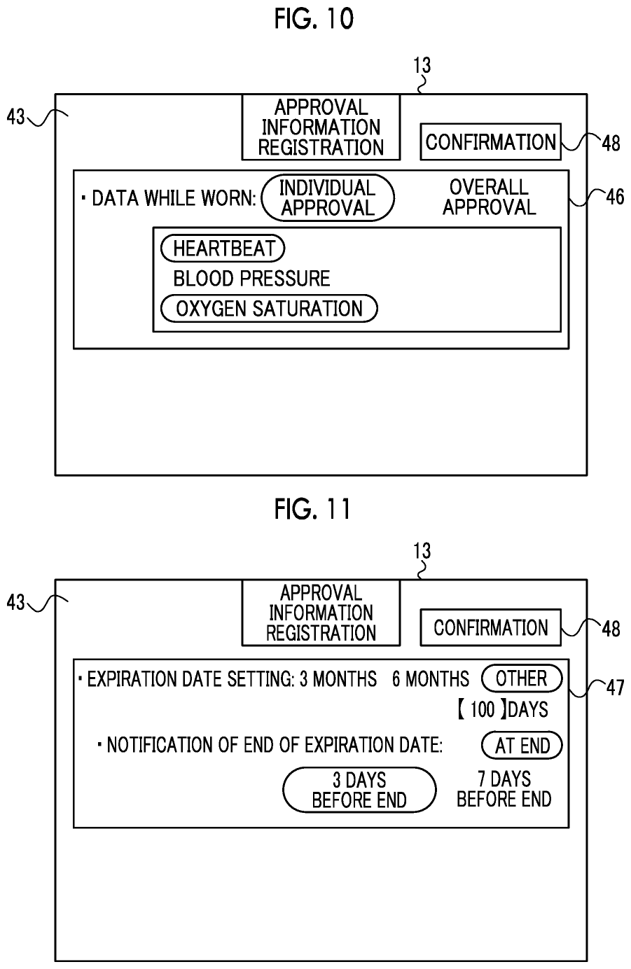

As shown in FIG. 10, in the provision data setting field 46, the measurement item of the data while worn to be provided is set. The smart watch 10 classifies the data while worn in a case of acquiring the data for each measurement item and stores the data in the measurement data storage unit 30. By selecting “individual approval” for the data while worn to be provided, it is possible to set whether or not to approve the data provision for each measurement item. The measurement items include, for example, a heartbeat, a blood pressure, an oxygen saturation, and the like and can be individually selected. The measurement items include, in addition to biological information such as a heartbeat and a blood pressure measured by each sensor, calorie consumption calculated from a plurality of pieces of biological information and the wearer information.

In addition, the setting of the measurement item of the data while worn to be provided may be set individually for each provision destination. For example, even in a case where the measurement item of the data while worn to be provided to the “normal diagnostic information generation” and the “machine learning diagnostic information generation” is “overall approval”, “individual approval” may be performed for the data while worn to be provided to the “re-training data”, and the contents of the data to be provided may be set in detail.

In the setting of the approval information, data expressing the wearer's intention to provide data for each measurement item may be stored, and the data may be collated with the request contents by the data collation unit 23 upon the data provision request to provide the data.

As shown in FIG. 11, an expiration date is set for the approval information. In the expiration date setting field 47, the approval information for which the expiration date is set is stored, and notification of expiration of the expiration date is given. In addition, an operation of updating the expiration date is received. The expiration date of, for example, 6 months is set in the initial state, and in a case where the expiration date has expired, notification of the end of the expiration date is given. A period of the expiration date and a timing of the notification can be arbitrarily set, and it may be set to give notification of the scheduled end of the expiration date 3 days or 7 days before the expiration date expires, in addition to the notification of the end of the expiration date. In addition, the expiration date of the approval information may be set in units of one day. For example, “100 days” is input with text input, or the expiration date is designated in conjunction with a calendar function. In a case where the expiration date has expired, all pieces of the approval information are reset. The wearer performs an update operation or reset of the prior approval information in response to the notification.

In FIGS. 8 to 11, in order to discriminate the selected item, highlighting is performed by surrounding the selected item with an ellipse. However, the selected item may be highlighted by another method such as changing a thickness or a color of a character. After the input is completed, the confirmation button 48 is selected, and the input setting contents are stored in the approval information storage unit 32 as the approval information. The selection of the confirmation button 48 may be performed in a case where the input is completed in each of the setting fields, or may be collectively performed in a case where the input on the approval information registration screen 43 is completed. In a case where the input in one setting field is completed and the input is performed in another setting field, the display of the display 13 is moved by a scroll operation including a swipe operation. A scroll bar (not shown) may be provided on the approval information registration screen 43 to receive the scroll operation.

The setting mode is switched to the normal mode after the setting on the approval information registration screen 43 in the setting mode is completed and the approval information is stored in the approval information storage unit 32. The smart watch 10 in the normal mode uses each function of the smart watch 10 and collects the data while worn of the wearer in a state in which the prior approval information set in the setting mode is stored. The wearer can perform biometric authentication at any timing from the normal mode as necessary and switch to the setting mode to display the approval information registration screen 43 and check or change the approval information. In addition, data under measurement stored in the measurement data storage unit 30 can be displayed on a screen and checked.

It is preferable that the data while worn is continuously collected from the start of collection and is stored in the measurement data storage unit 30. On the other hand, in order to reduce pressure on data capacity, the data while worn for only the latest 24 hours or 48 hours may be stored in the measurement data storage unit 30.

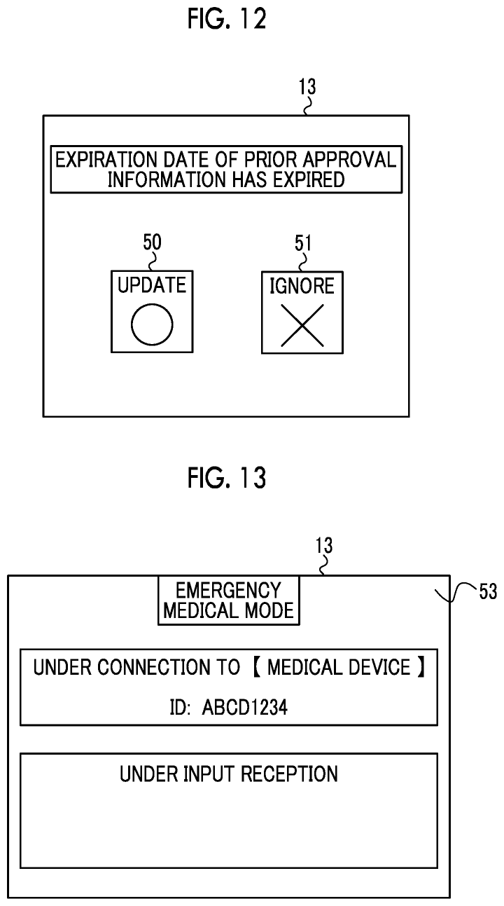

As shown in FIG. 12, in a case of performing the notification at the end of the set expiration date in the normal mode, a message indicating the end of the expiration date, an update button 50, and an ignore button 51 are displayed on the display 13. The update button 50 and the ignore button 51 receive the selection of the wearer, and in a case where the update button 50 is selected, the biometric authentication information is requested, and the expiration date is updated in a case where the wearer is determined to be the person in question in the biometric authentication. In a case where there is no change in the expiration date after the update, the confirmation button 48 is selected as it is, and the screen returns to the screen before the notification in the normal mode. In a case where the wearer selects the ignore button 51, the approval information is reset without updating the expiration date, and the screen returns to the screen before the notification. The reset approval information is manually set again by the wearer in the setting mode.

In a case of performing the notification before the end of the expiration date, a message indicating the scheduled end of the expiration date, a scheduled end date, the update button 50, and the ignore button 51 are displayed. In a case where the update button 50 is selected, the update is performed in the same manner as the notification at the end of the expiration date, and in a case where the ignore button 51 is selected, the update is not performed and the notification is performed again at the end of the expiration date.

The emergency medical mode is used in a medical emergency of the wearer, for example, in a case where the wearer loses consciousness due to an injury or sudden illness while going out and falls down and is taken to a medical facility such as the nearest emergency hospital. The switching to the emergency medical mode is made, for example, by a specific operation by a medical worker or an abnormality detection of the smart watch 10. In the emergency medical mode, it is preferable to automatically collect and store the data while worn regardless of setting of a storage range of the data while worn in the normal mode.

The specific operation is a connection by a device such as a contactless device (not shown) that is compatible with the smart watch 10 and can be recognized in a non-contact manner or an input of a specific command to the smart watch 10. The contactless device is, for example, an integrated circuit (IC) chip reader connected by wire to a PC in a medical facility, and recognizes an IC chip provided in the smart watch 10 by bringing the smart watch 10 close to the contactless device by a certain distance. Upon recognition, the smart watch 10 is switched to the emergency medical mode and automatically connected. In addition, in a case where the smart watch 10 is connected, a wearable device manufacturer has information unique to each medical facility that is distributed and managed for each medical facility, particularly for each emergency hospital. In addition, the contactless device may be a two-dimensional code reader connected to a PC of a medical facility, and may be connected by being displayed on the display 13 and reading a two-dimensional code along with switching of the smart watch 10 to the emergency medical mode.

In a case where the smart watch 10 is connected to the contactless device, it is determined whether or not a patient has previously approved the provision of data to the medical facility. The determination is made based on the collation result of the data collation unit 23 as to whether or not the smart watch 10 stores information unique to each medical facility and the like as the medical device information in the external device information storage unit 33 in a case where the smart watch 10 is connected, and whether or not the stored medical device information is included in the data provision destination in the “provision destination setting” in the approval information. For example, in a case where the facility name of the medical facility acquired as the medical device information is included in the “provision destination setting” or in a case where the “provision destination setting” is set to “all medical facilities”, the smart watch 10 determines that the data provision is approved and receives the data provision request.

In addition, in a case where the biological information sensor 16 detects an abnormality of the wearer, switching may be performed automatically. The abnormality detection is, for example, detection of an excessive or unstable heartbeat, and acquisition of data such as a blood pressure that does not fall within a predetermined range or a decrease in a blood oxygen concentration. In this case, in a case of switching to the emergency medical mode due to the abnormality detection, the data while worn for the latest several minutes may always be temporarily stored and the temporarily stored data while worn may be permanently stored in a case where the abnormality is detected, as in a drive recorder.

As shown in FIG. 13, in a case where the smart watch 10 in the emergency medical mode is connected to a medical device, it is preferable that an emergency medical mode screen 53 is deployed on the display 13 to display connection information. The connection information is a name of a connected medical device, an ID thereof, or the like, and it is sufficient as long as the medical worker can discriminate the device to be connected at a glance. The connection information may be product information of the medical device 35 or may be set independently at the medical facility.

The smart watch 10 under connection to the medical device 35 receives the data provision request from the medical device 35, and collates the data provision request with the approval information. In accordance with the collation result, the data provision is performed such that the data while worn included in the data provision request and approved for provision in the approval information is transmitted to the medical device 35. Regarding the data provision, for example, in addition to measured data such as heartbeat and blood pressure records for the last 24 hours, data such as date of birth, blood type, and medical history stored in the smart watch 10 by the wearer in advance may also be provided in response to the data provision request. Unless otherwise specified, the request contents of the data provision request include requesting all pieces of the biological information measured by the smart watch 10.

The smart watch 10 under connection to the medical device 35 may perform the data provision request not from the medical device 35 but by a medical worker operating the smart watch 10. In this case, the medical worker performs authentication for preventing impersonation. For example, a smart watch manufacturer may store the face and fingerprint of a medical worker in a database (server) or the like in advance, and transmit the data once the medical facility name and the medical worker are authenticated.

After the data is provided, the mode is switched to the normal mode after a lapse of a certain period or by a user operation. In addition, even for the content that is included in the data provision request and approved for provision in the approval information, in a case where the content is not the data stored in the measurement data storage unit 30, it is preferable to leave it as it is by displaying a message to that effect or the like.

In a case where the smart watch 10 does not store the data while worn, or in a case where there is no data while worn that can be provided by collating the approval information with the medical device information, or in a case where the medical worker cannot be authenticated, a message indicating that the data cannot be provided may be displayed. The collation result between the approval information and the data provision request includes a case where the approval information is not registered.

The smart watch 10 that is in the emergency medical mode and connected to the medical device 35 acquires the medical device information including affiliation information and the usage of the data while worn from the medical device 35. The smart watch 10 that has received the data provision request collates the data while worn that can be provided based on the approval information and the medical device information, and provides the data while worn to the medical device 35. The order of acquisition of the medical device information and reception of the data provision request may be either first or may be simultaneous.

The determination to provide the data while worn according to the affiliation information in the acquired medical device information is made in a case where the destination of the data provision is designated in the approval information. For example, as shown in FIG. 9, in a case where the provision destinations of the data while worn are designated to “ABC hospital” and “XYZ clinic”, and the acquired affiliation information matches the content of the provision destination designation at the “ABC hospital”, the data while worn is provided according to the collation result between the other approval information and the content of the data provision request. In a case where the acquired affiliation information is “DEF hospital” and does not match the content of the provision destination designation, the data is not provided. In a case where the provision destination designation is “None”, the data can be provided regardless of the affiliation information. It is preferable that the determination as to whether or not to provide the data while worn according to the affiliation information is made before collation of the request content of the data provision request.

In addition, from the acquired medical device information, it is discriminated whether or not the medical device 35 generates the machine learning diagnostic information regarding the usage of the data while worn in the medical device 35 of the provision destination. In a case where the medical device 35 generates the machine learning diagnostic information, the data provision request may include a request for providing the data while worn for the generation of the medical diagnostic information using machine learning (machine learning diagnostic information) and a request for providing the data while worn used for the re-training process in the machine learning. Therefore, the smart watch 10 provides the data while worn for the generation of the medical diagnostic information using machine learning in a case where “machine learning diagnostic information generation” is approved for the usage of the approval information, as the setting contents of the approval information are shown in FIG. 8. In addition, in a case where the “re-training data” is approved for the usage, the data is provided for the re-training process in the machine learning.

The data provision request for the generation of the medical diagnostic information and the data provision request for the re-training process of the machine learning may be performed simultaneously or individually. In a case where the data provision requests are performed simultaneously, and the prior approval information approves only the medical treatment, it is communicated that the data while worn to be provided is not approved for use in the re-training process of the machine learning by displaying on the display 13 or the like. Alternatively, the data while worn to be provided is tagged with a wording such as “for medical purposes only” or the wording is added to a data name.

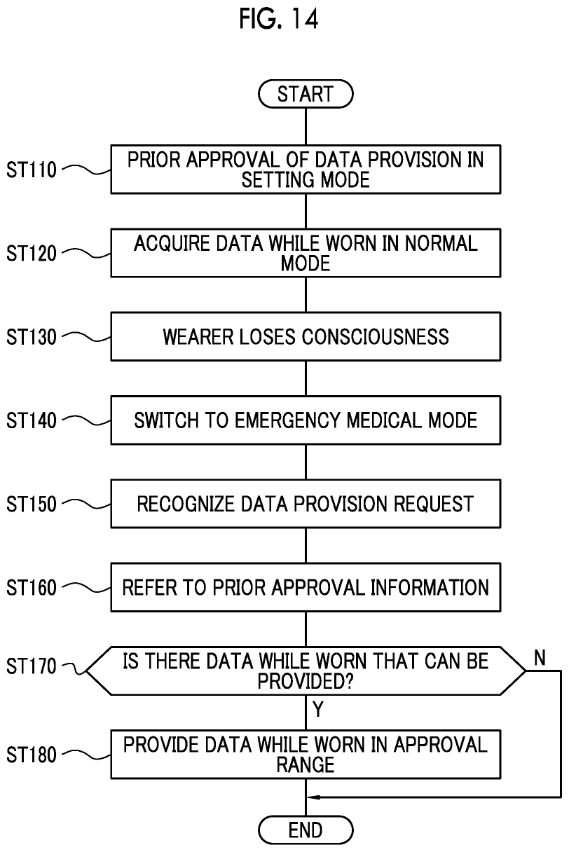

Next, a series of flows of providing biological data recorded while wearing the smart watch 10 to the medical worker even in a case where a user is in a state such as loss of consciousness will be described with reference to a flowchart shown in FIG. 14.

The smart watch 10 receives a prior approval operation by the wearer and stores in advance registration of the approval information for approving the provision of the data while worn of the wearer to the medical facility in an emergency (step ST110). The smart watch 10 after the prior approval operation collects and stores the data while worn including at least biological information in a healthy state of the wearer in the normal mode (step ST120). The wearer loses consciousness due to illness, injury, or the like and is transported to the medical facility (step ST130). An abnormality in the biological information acquired by the biological information sensor 16 may be detected in a case where the wearer loses consciousness.

The smart watch 10 switches from the normal mode to the emergency medical mode by detecting an abnormality by the biological information sensor 16 or an input operation or the like by a medical worker or the like at a transport destination (step ST140). The smart watch 10 in the emergency medical mode receives the data provision request to provide the stored data while worn to the medical device 35 which generates the medical diagnostic information, by a specific operation of the medical device 35 provided in the medical facility, the medical worker, or the like (step ST150). The smart watch 10 collates the content of the data provision request with the approval information stored in advance (step ST160). Based on the collation result, the presence or absence of the data while worn that can be provided is selected (step ST170).

In a case where there is the data while worn that can be provided (Y in step ST170), the data while worn is provided to the medical device 35 in response to the data provision request (step ST180). After the data is provided, the mode is switched to the normal mode after a lapse of a certain period or by a user operation.

In a case where there is no biological information that can be provided (N in step ST170), it is preferable to switch to the normal mode.

As a modification example of the first embodiment, a case where the approval information is created using proxy approval by a proxy different from the wearer instead of prior approval will be described. The proxy stores the biometric authentication information of the proxy in the biometric authentication information storage unit 31 in advance in the setting mode. The proxy is a family member or guardian of the wearer, or a relative, partner, or close friend of the wearer. For example, in a case where the wearer is a minor and the guardian manages personal information of the wearer, the guardian approves the registration of the approval information as a proxy on behalf of the wearer.

The proxy registers the biometric authentication information as the wearer does at a timing such as at the time of initial setting, and registers the approval information from the approval information registration screen in a state in which the biometric authentication is enabled. The approval information registered by the proxy may be set such that editing is not accepted even though the approval information can be viewed by the wearer himself/herself.

Further, the proxy approval may be performed by storing the approval information in the approval information storage unit 32 as the proxy approval in advance. However, even in a state in which there is no approval information registered in advance, the proxy approval may be performed in real time as long as the biometric authentication enables the proxy to be identified, and the data while worn may be provided in response to the data provision request. For example, in a case where the wearer is transported to an emergency hospital or the like, a guardian may accompany the wearer to operate the smart watch 10 at a medical facility to create the approval information.

The approval information does not have to be disclosed to the medical worker even in a case of providing the data. For example, only the wearer and the proxy who have registered the biometric authentication information may be able to view the approval information displayed on a screen of the display 13.

### Second Embodiment

In the second embodiment, the smart watch 10 provides data using the approval information created by an external input device 60 such as a PC or a smartphone, instead of registering the approval information in advance in the setting mode. The external input device 60 may be operated by the wearer himself/herself, or may employ a proxy approval operated by a proxy different from the wearer. Other details are the same as those of the first embodiment.

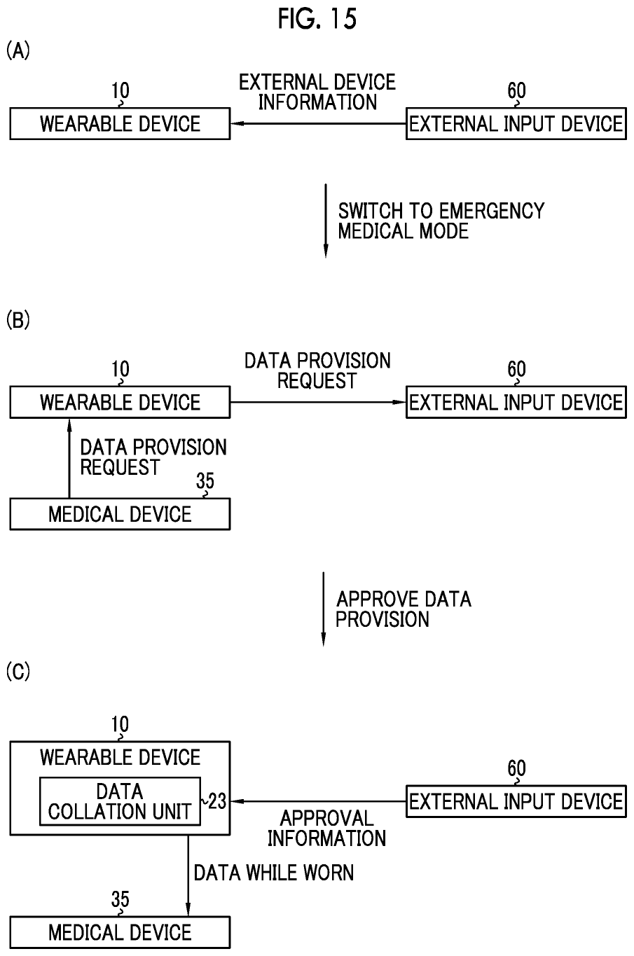

As shown in FIG. 15, in the second embodiment, in the setting mode, the smart watch 10 registers in advance the external input device 60 capable of creating and transmitting the approval information in the external device information storage unit 33 (see (A) in FIG. 15). In the emergency medical mode, the smart watch 10 transfers the data provision request to the external input device 60 in response to the reception of the data provision request from the medical device 35 (see (B) in FIG. 15). The external input device 60 creates the approval information by receiving the data provision request. The smart watch 10 receives the approval information from the external input device 60, collates the approval information with the data provision request and the stored data under measurement by the data collation unit 23, selects the data while worn that can be provided, and provides the data while worn to the medical device 35 (see (C) in FIG. 15).

In the creation and transmission of the approval information in the external input device 60, for example, the external input device 60 connects to an access authentication server via the Internet, at least sets access permission for each item of the data while worn, and transmits the setting content as approval information to the smart watch 10. In this case, the smart watch 10 receives the approval information from the access authentication server.

Since the approval using the external input device 60 uses a user interface or a large screen that is easier to operate than the smart watch 10, even in a state in which the wearer does not lose consciousness at the medical facility or the like of the transport destination and can consent to the provision of data, it is easier to reduce operation errors and to check the content of the screen display, so that data can be provided promptly. In addition, in a case of proxy approval, the proxy does not need to be in the same place as the wearer, and can remotely operate the external input device 60 to approve the data provision. For example, in a case where the wearer loses consciousness and is transported to an emergency hospital or the like, a guardian who has been contacted performs proxy approval remotely from his/her own information terminal.

### Third Embodiment

In the third embodiment, instead of the data storage unit 22, the data while worn is uploaded and stored in an external storage device 70 such as a server, and in the emergency medical mode, the medical device 35 in the medical facility receives the data provision from the external storage device 70. The data while worn may be stored on a cloud. Other details are the same as those of the first embodiment.

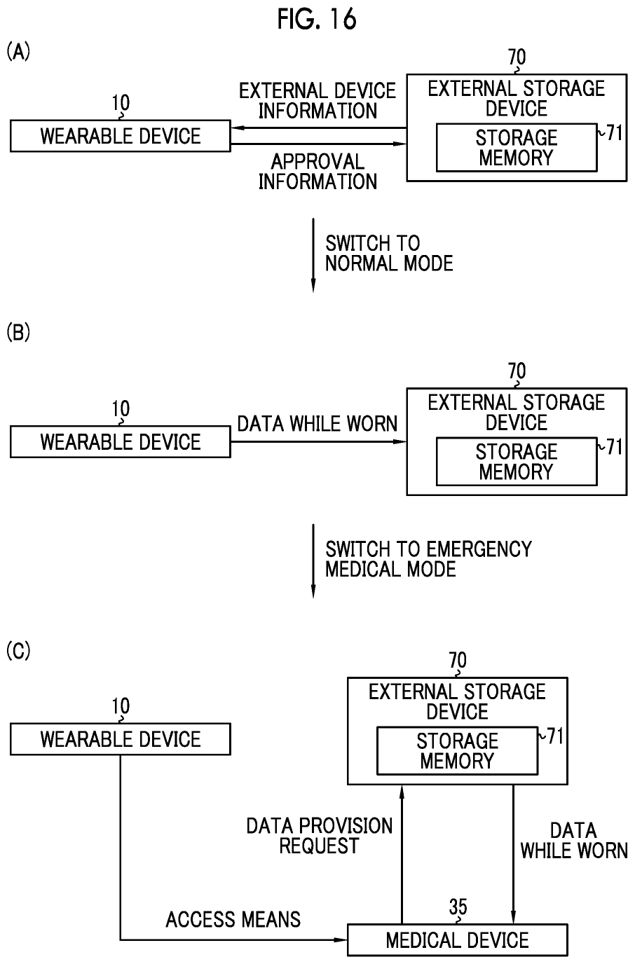

As shown in FIG. 16, in the third embodiment, the smart watch 10 in the setting mode registers the external storage device 70 having a storage memory 71 that realizes a storage function, and enables data transfer (see (A) in FIG. 16). From the smart watch 10 in the normal mode, the external storage device 70 receives the latest data while worn and stores the data while worn in the storage memory 71 (see (B) in FIG. 16). The data while worn may be transmitted manually or automatically. The smart watch 10 in the emergency medical mode transmits access means and access permission to the external storage device 70 at the medical facility of the transport destination, and the medical device 35 connects to the external storage device 70 to transmit the data provision request. The external storage device 70 collates the received data provision request with the stored approval information, and provides the medical device 35 with the data while worn stored in the storage memory 71 according to the collation result (see (C) in FIG. 16).

In the setting mode, the smart watch 10 is set to store information on the external storage device 70 in the external device information storage unit 33 via the communication unit 25 and to transmit and store the data while worn to the external storage device 70. In a case where the data while worn is automatically stored, a frequency of data storage is set every fixed period such as, for example, 3 days. In addition, the approval information stored in the approval information storage unit 32 is transmitted to the external storage device 70 and stored. It is preferable that the external storage device 70 includes a dedicated site that serves as an interface in a case of being connected from a certain device. The smart watch 10 switches from the setting mode to the normal mode as soon as the acquisition of the external device information and the transmission of the approval information are completed.

In the normal mode, the smart watch 10 collects the data while worn of the wearer and automatically or manually transmits the data while worn to the external storage device 70. The transmitted data while worn may be deleted from the data storage unit 22. For example, a measurement unit deletes the data while worn that is old or deletes the data while worn that is one month old from a measurement date in response to the pressure on the data capacity. In addition, it is also possible to check the data while worn stored in the external storage device 70 from the smart watch 10 as necessary.

In the emergency medical mode, in a case where the smart watch 10 receives a specific operation by a medical worker, the access to the external storage device 70 is permitted, and the access means to the external storage device 70 is displayed on the screen. The access permission is to issue permission of access from the medical device 35 in a temporary period such as 60 minutes after receiving the specific operation. In the screen display of the access means, a two-dimensional code including information such as a uniform resource locator (URL) to the external storage device 70 or a wearer ID is displayed. The screen may be displayed on the display 13 or may be displayed on the medical device 35 connected to the smart watch 10. In addition, in a case where the wearer has not lost consciousness, instead of the specific operation of the medical worker, the wearer may perform biometric authentication and directly present the access permission and the access means.

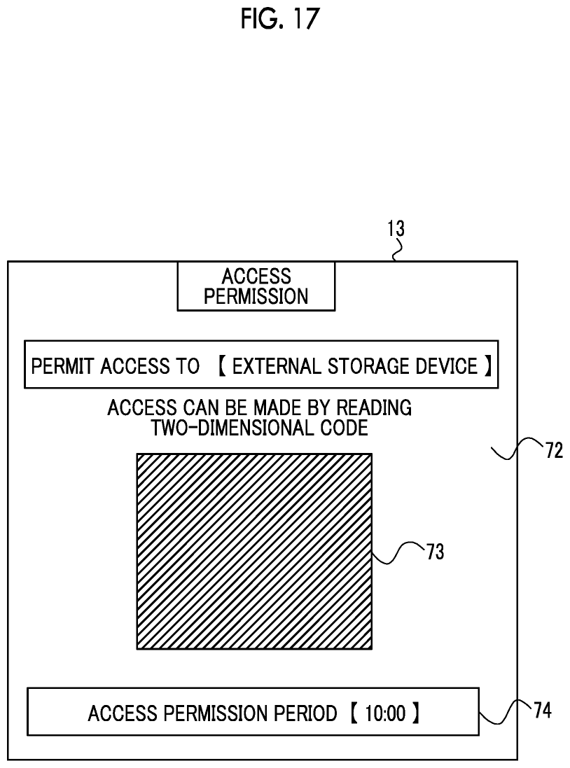

As shown in FIG. 17, as the access means, an access permission screen 72 is deployed on the display 13 to display a two-dimensional code 73 and an access permission period display field 74 representing an access permission period by countdown. The medical worker can connect to a dedicated site that accesses the external storage device 70 by reading the two-dimensional code 73. It is preferable that the two-dimensional code 73 includes a URL to the external storage device 70, an ID of the wearer, approval information, and a summary of the stored data while worn. In addition, the connection to the dedicated site may be made by means of displaying character strings of a URL, an ID, or approval information on a screen or transmitting them to the medical device 35. It may be determined whether or not the data while worn required by the medical worker can be acquired from the approval information and the summary of the data while worn acquired by reading the two-dimensional code 73. The access permission period may be set to any period in a case of setting the approval information. For example, it is preferable to set the period such as 10 minutes.

In the dedicated site, it is preferable to link the biometric authentication of the medical worker with an identification of a hospital name. In a case where the biometric authentication using the face or fingerprint of the medical worker is received and the hospital name is identified, the external storage device 70 controls the data provision based on the approval information and the data provision request. Accordingly, it is possible to provide the data while worn to the medical device 35 in a state in which the data while worn stored in the smart watch 10 is backed up or a large amount of data that cannot be stored in the smart watch 10 is stored.

The smart watch 10 stores the data while worn before storage in the external storage device 70. Therefore, it is possible to acquire the data while worn from the smart watch 10 in combination with the first embodiment.

In addition, the external input device 60 that creates and transmits the approval information in the second embodiment may be combined with the external storage device 70 that stores the data while worn in the third embodiment. In such a case, the external input device 60 and the external storage device 70 may be the same server or the like.

The wearable device is not limited to the smart watch 10, and may be a terminal worn on the finger, a terminal worn on the neck, or a combination thereof as long as it has a function of realizing the above-described embodiments.

In the above-described embodiments, hardware structures of the processing units that execute various processes such as the data acquisition unit 20, the data analysis unit 21, the data storage unit 22, the data collation unit 23, the output control unit 24, the communication unit 25, the input reception unit 26, and the mode switching unit 27 are various processors as described below. Examples of the various processors include a central processing unit (CPU) which is a general-purpose processor that executes software (program) to function as various processing units, a graphical processing unit (GPU), a programmable logic device (PLD) which is a processor of which a circuit configuration is changeable after manufacture, such as a field programmable gate array (FPGA), and a dedicated electrical circuit which is a processor having a dedicated circuit configuration specially designed to execute various processes.

One processing unit may be composed of one of the various processors or may be composed of a combination of two or more processors of the same type or different types (for example, a combination of a plurality of FPGAs, a combination of a CPU and an FPGA, or a combination of a CPU and a GPU). In addition, a plurality of processing units may be configured by one processor. As an example in which the plurality of processing units are composed of one processor, first, there is a form in which one processor is composed of a combination of one or more CPUs and software, and this processor functions as the plurality of processing units, as typified by a computer, such as a client or a server. Second, there is a form in which a processor that realizes functions of an entire system including a plurality of processing units with one integrated circuit (IC) chip is used, as typified by a system on chip (SoC) or the like. As described above, the various processing units are configured using one or more of the above various processors as a hardware structure.

Furthermore, the hardware structures of these various processors are more specifically electrical circuitry in a form in which circuit elements, such as semiconductor elements, are combined. A hardware structure of the storage unit is a storage device such as a hard disc drive (HDD) or a solid state drive (SSD).

### EXPLANATION OF REFERENCES

10: smart watch
11: smart watch main body
11a: main body front surface
11b: main body back surface
12: belt
13: display
14: camera
15: operation button
16: biological information sensor
17: light emitting portion
18: light receiving portion
20: data acquisition unit
21: data analysis unit
22: data storage unit
23: data collation unit
24: output control unit
25: communication unit
26: input reception unit
27: mode switching unit
30: measurement data storage unit
31: biometric authentication information storage unit
32: approval information storage unit
33: external device information storage unit
35: medical device
40: biometric authentication screen
41: authentication method selection field
43: approval information registration screen
44: usage setting field
45: provision destination setting field
46: provision data setting field
47: expiration date setting field
48: confirmation button
50: update button
51: ignore button
53: emergency medical mode screen
60: external input device
70: external storage device
71: storage memory
72: access permission screen
73: two-dimensional code
74: access permission period display field
ST: step

What is claimed is:

1. A wearable device comprising:
one or more processors configured to:
collect data while worn including at least biological information of a wearer in a healthy state;
store approval information for controlling output of the data while worn by prior approval of the wearer;
receive a data provision request for a medical device that generates medical diagnostic information; and
provide the data while worn to the medical device based on the approval information and the data provision request.

2. The wearable device according to claim 1, wherein the one or more processors are configured to:
classify the data while worn for each measurement item; and
store the approval information in which whether or not to provide data is set for each measurement item.

3. The wearable device according to claim 1, wherein the one or more processors are configured to:
store the approval information in which an expiration date is set;
give a notification at an end of the expiration date; and
receive an update operation of the expiration date.

4. The wearable device according to claim 1, wherein the one or more processors are configured to:
acquire medical device information from the medical device; and
provide the data while worn to the medical device based on the approval information and the medical device information.

5. The wearable device according to claim 4, wherein the one or more processors are configured to:
acquire affiliation information of the medical device from the medical device information; and
provide the data while worn according to the affiliation information.

6. The wearable device according to claim 4, wherein the one or more processors are configured to:
discriminate the medical device for performing machine learning from the medical device information; and
provide the data while worn for generation of the medical diagnostic information using the machine learning.

7. The wearable device according to claim 4, wherein the one or more processors are configured to:
discriminate the medical device for performing machine learning from the medical device information; and
provide the data while worn for a re-training process in the machine learning.

8. The wearable device according to claim 1, wherein the one or more processors are configured to:
perform biometric authentication of the wearer; and
receive the prior approval in a case where the wearer is determined by the biometric authentication.

9. The wearable device according to claim 8, wherein the one or more processors are configured to display the approval information on a screen by the biometric authentication.

10. The wearable device according to claim 1, wherein the one or more processors are configured to create the approval information using proxy approval by a proxy different from the wearer, instead of the prior approval.

11. The wearable device according to claim 1, wherein the one or more processors are configured to:
register in advance an external input device capable of creating the approval information;
receive the approval information from the external input device; and
provide the data while worn.

12. The wearable device according to claim 1, wherein the one or more processors are configured to:
store the data while worn in an external storage device; and
provide the data while worn to the medical device from the external storage device in response to the approval information and the data provision request.

13. The wearable device according to claim 12, wherein the one or more processors are configured to permit access to the external storage device and display access means on a screen by biometric authentication or a specific operation by a medical worker based on the approval information.

14. A method of operating a wearable device, comprising:
a step of collecting data while worn including at least biological information of a wearer in a healthy state;
a step of storing approval information for controlling output of the data while worn by prior approval of the wearer;
a step of receiving a data provision request for a medical device that generates medical diagnostic information; and
a step of providing the data while worn to the medical device based on the approval information and the data provision request.

15. A non-transitory computer readable medium for storing a computer-executable program for causing a computer to function as a wearable device, the computer-executable program causing the computer to execute:
a function of collecting data while worn including at least biological information of a wearer in a healthy state;
a function of storing approval information for controlling output of the data while worn by prior approval of the wearer;
a function of receiving a data provision request for a medical device that generates medical diagnostic information; and
a function of providing the data while worn to the medical device based on the approval information and the data provision request.
